(19) United States
(12) Patent Application Publication
(10) Pub. No.: US 2003/0091598 A1
(43) Pub. Date: May 15, 2003

(54) Compounds and methods for the diagnosis and treatment of Babesia infection

(75) Inventors: Mary J. Homer, Seattle, WA (US); Michael J. Lodes, Seattle, WA (US); Raymond L. Houghton, Bothell, WA (US); David H. Persing, Redmond, WA (US); Patricia D. McNeill, Federal Way, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/234,432

(22) Filed: Aug. 30, 2002

Related U.S. Application Data
(63) Continuation-in-part of application No. PCT/US02/00026, filed on Jan. 4, 2002.
(60) Provisional application No. 60/325,097, filed on Sep. 25, 2001.
(60) Provisional application No. 60/269,240, filed on Feb. 15, 2001.
(60) Provisional application No. 60/260,246, filed on Jan. 5, 2001.

(51) Int. Cl. A61K 39/018; C12Q 1/68; C07H 21/04; C12N 1/10; C12N 9/00; C12N 15/74

(52) U.S. Cl. 424/270.1; 435/6; 435/69.3; 435/183; 435/320.1; 435/258.1; 536/23.7

(57) ABSTRACT

Compounds and methods for the diagnosis and treatment of Babesia sp. WA1 infection are disclosed. The compounds provided include polypeptides that contain at least one immunogenic portion of a Babesia sp. WA1 antigen and polynucleotides encoding such polypeptides. Pharmaceutical compositions and immunogenic compositions comprising such polypeptides or polynucleotides are also provided. Diagnostic kits containing such polypeptides or polynucleotides and a suitable detection reagent may be used for the detection of Babesia sp. WA1 infection in patients and biological samples. Antibodies directed against such polypeptides are also provided.

### CROSS REFERENCE TO RELATED APPLICATIONS

This patent application is a continuation-in-part application of International Application PCT/US02/00026, now pending, with an international filing date of Jan. 4, 2002, published in English under PCT Article 21(2), which claims priority to U.S. Provisional Application No. 60/260,246 filed Jan. 5, 2001, U.S. Provisional Application No. 60/269,240, filed Feb. 15, 2001, and U.S. Provisional Application No. 60/325,097, filed Sep. 25, 2001, incorporated in their entirety herein.

### STATEMENT OF GOVERNMENT INTEREST

This invention was made in part with funds provided by the United States Government under National Institutes of Health Grant AI-36810. Accordingly, the United States Government may have certain rights to this invention.

### BACKGROUND OF THE INVENTION

1. Field of the Invention

The present invention relates generally to the detection of infection with the Babesia species referred to as WA1. In particular, the invention is related to polypeptides comprising Babesia sp. WA1 antigens and the use of such polypeptides for the serodiagnosis and treatment of infection with the Babesia sp. WA1.

2. Description of the Related Art

Babesiosis is an intraerythrocytic protozoan infection that results in a malaria-like illness. Babesiosis is most frequently attributed to infection with the rodent parasite Babesia microti (B. microti) which is generally transmitted to humans by the same tick that is responsible for the transmission of Lyme disease and ehrlichiosis, thereby leading to the possibility of co-infection with babesiosis, Lyme disease and ehrlichiosis from a single tick bite. The number of reported cases of Babesia infection in the United States is increasing rapidly, with most cases being located in the eastern states. However, infection with a previously unknown species of Babesia, referred to as WA1, has been reported in the western US (Quick et al. Ann. Internal Med. (1993) 119:284-290; and Thomford et al. Jnl. Infect. Dis. (1994) 169:1050-1056). Babesia sp. WA1 has been found to be antigenically and genotypically distinct from B. microti. Furthermore, infection with Babesia sp. WA1 leads to more severe disease than infection with B. microti.

Infection with Babesia species, including co-infection with Lyme disease, often remains undetected for extended periods of time. Babesiosis is potentially fatal, particularly in the elderly and in patients with suppressed immune systems. Patients infected with both Lyme disease and babesiosis have more severe symptoms and prolonged illness compared to those with either infection alone.

The preferred treatments for Lyme disease, ehrlichiosis and babesiosis are different, with penicillins, such as doxycycline and amoxicillin, being most effective in treating Lyme disease, tetracycline being preferred for the treatment of ehrlichiosis, and anti-malarial drugs, such as quinine and clindamycin, being most effective in the treatment of babesiosis. Accurate and early diagnosis of Babesia infection is thus critical but methods currently employed for diagnosis are problematic. All three tick-borne illnesses share the same flu-like symptoms of muscle aches, fever, headaches and fatigue, thus making clinical diagnosis difficult. Microscopic analysis of blood samples may provide false-negative results when patients are first seen in the clinic. Indirect fluorescent antibody staining methods for total immunoglobulins to B. microti may be used to diagnose infection with B. microti, but such methods are time-consuming and expensive. There thus remains a need in the art for improved methods for the detection of Babesia infection.

### BRIEF SUMMARY OF THE INVENTION

The present invention provides compositions and methods for the diagnosis and treatment of infection with Babesia sp. WA1.

In one aspect, the present invention provides polynucleotide compositions comprising a sequence selected from the group consisting of: (a) sequences provided in SEQ ID NO: 1-23, 40-56, 72-74, 85 and 87; (b) complements of the sequences provided in SEQ ID NO: 1-23, 40-56, 72-74, 85 and 87; (c) sequences that hybridize to a sequence provided in SEQ ID NO: 1-23, 40-56, 72-74, 85 and 87, under moderately stringent conditions; (e) sequences having at least 75% identity to a sequence of SEQ ID NO: 1-23, 40-56, 72-74, 85 and 87; (f) sequences having at least 90% identity to a sequence of SEQ ID NO: 1-23, 40-56, 72-74, 85 and 87; and (g) degenerate variants of a sequence provided in SEQ ID NO: 1-23, 40-56, 72-74, 85 and 87.

In a related aspect, expression vectors comprising such polynucleotides and host cells transformed or transfected with such expression vectors are also provided.

In another aspect, polypeptides are provided comprising an immunogenic portion of a Babesia sp. WA1 antigen, or a variant thereof, wherein the immunogenic portion is encoded by one of the inventive polynucleotides. In specific embodiments, polypeptide comprising an amino acid sequence selected from the group consisting of SEQ ID NO: 24-39, 57-71, 75-77, 84, 86 and 88 are provided.

In another aspect, the present invention provides fusion proteins comprising either a first and a second inventive polypeptide, or, alternatively, an inventive polypeptide and a known antigen.

In further aspects of the subject invention, methods and diagnostic kits are provided for detecting Babesia sp. WA1 infection in a patient. In one embodiment, the method comprises: (a) contacting a biological sample with at least one polypeptide of the present invention; and (b) detecting in the sample the presence of antibodies that bind to the polypeptide, thereby detecting Babesia sp. WA1 infection in the biological sample. In other embodiments, the methods comprise: (a) contacting a biological sample with at least one of the above polypeptides; and (b) detecting in the sample the presence of antibodies that bind to the polypeptide. Suitable biological samples include whole blood, sputum, serum, plasma, saliva, cerebrospinal fluid and urine. Diagnostic kits for use in such methods comprise one or more of the above polypeptides in combination with a detection reagent.

The present invention also provides methods for detecting Babesia sp. WA1 infection comprising: (a) obtaining a biological sample from a patient; (b) contacting the sample with at least two oligonucleotide primers in a polymerase chain reaction, wherein at least one of the oligonucleotide primers is specific for a DNA sequence encoding the above polypeptides; and (c) detecting in the sample a DNA sequence that amplifies in the presence of the first and second oligonucleotide primers. In one embodiment, the oligonucleotide primer comprises at least about 10 contiguous nucleotides of a polynucleotide encoding the above polypeptides.

In a further aspect, the present invention provides a method for detecting Babesia sp. WA1 infection in a patient comprising: (a) obtaining a biological sample from the patient; (b) contacting the sample with an oligonucleotide probe specific for a polynucleotide encoding the above polypeptides; and (c) detecting in the sample a DNA sequence that hybridizes to the oligonucleotide probe. In one embodiment of this aspect, the oligonucleotide probe comprises at least about 15 contiguous nucleotides of a polynucleotide encoding the above polypeptides.

In yet another aspect, the present invention provides antibodies, both polyclonal and monoclonal, that bind to the polypeptides described above, as well as methods for their use in the detection of Babesia sp. WA1 infection.

Within other aspects, the present invention provides pharmaceutical compositions that comprise one or more of the above polypeptides, or polynucleotides and a physiologically acceptable carrier. The invention also provides immunogenic compositions comprising one or more of the inventive polypeptides or polynucleotides and an immunostimulant.

In yet another aspect, methods are provided for inducing protective immunity in a patient, comprising administering to a patient an effective amount of one or more of the above pharmaceutical or immunogenic compositions.

These and other aspects of the present invention will become apparent upon reference to the following detailed description. All references disclosed herein are hereby incorporated by reference in their entirety as if each was incorporated individually.

Sequence Identifiers

SEQ ID NO: 1 is the determined cDNA sequence for clone 60s.2.

SEQ ID NO: 2 is the determined cDNA sequence for clone BiP4.

SEQ ID NO: 3 is the determined cDNA sequence for clone BiP57.

SEQ ID NO: 4 is the determined cDNA sequence for clone BiP63.

SEQ ID NO: 5 is the determined cDNA sequence for clone BiP80.

SEQ ID NO: 6 is the determined cDNA sequence for clone HSP70.19.

SEQ ID NO: 7 is the determined cDNA sequence for clone HSP70.22.

SEQ ID NO: 8 is the determined cDNA sequence for clone HSP70.28.

SEQ ID NO: 9 is the determined cDNA sequence for clone HSP70.61.

SEQ ID NO: 10 is the determined cDNA sequence for clone HSP70.68.

SEQ ID NO: 11 is the determined cDNA sequence for clone HSP70.94.

SEQ ID NO: 12 is the determined cDNA sequence for clone WA11.

SEQ ID NO: 13 is the determined cDNA sequence for clone WA16.

SEQ ID NO: 14 is the determined cDNA sequence for clone WA1.

SEQ ID NO: 15 is the determined cDNA sequence for clone WA24.

SEQ ID NO: 16 is the determined cDNA sequence for clone WA33.

SEQ ID NO: 17 is the determined cDNA sequence for clone WA38.

SEQ ID NO: 18 is the determined cDNA sequence for clone WA41.

SEQ ID NO: 19 is the determined cDNA sequence for clone WA5.

SEQ ID NO: 20 is the determined cDNA sequence for clone WA6.

SEQ ID NO: 21 is the determined cDNA sequence for clone WA74.

SEQ ID NO: 22 is the determined cDNA sequence for clone WA75.

SEQ ID NO: 23 is the determined cDNA sequence for clone WA76.

SEQ ID NO: 24 is the N-terminal amino acid sequence for clone WA75.

SEQ ID NO: 25 is the amino acid sequence for clone WA74.

SEQ ID NO: 26 is the amino acid sequence for clone WA5.

SEQ ID NO: 27 is the amino acid sequence for clone WA41.

SEQ ID NO: 28 is the amino acid sequence for clone WA38.

SEQ ID NO: 29 is the amino acid sequence for clone WA33.

SEQ ID NO: 30 is the amino acid sequence for clone WA1.

SEQ ID NO: 31 is the amino acid sequence for clone WA16.

SEQ ID NO: 32 is the amino acid sequence for clone WA11.

SEQ ID NO: 33 is the amino acid sequence for clone HSP70.94.

SEQ ID NO: 34 is the amino acid sequence for clone HSP70.68.

SEQ ID NO: 35 is the amino acid sequence for clone HSP70.28.

SEQ ID NO: 36 is the amino acid sequence for clone HSP70.19.

SEQ ID NO: 37 is the amino acid sequence for clone BiP57.

SEQ ID NO: 38 is the amino acid sequence for clone BiP4.

SEQ ID NO: 39 is the amino acid sequence for clone 60s.2.

SEQ ID NO: 40 is the determined cDNA sequence for clone WA01.

SEQ ID NO: 41 is the determined cDNA sequence for clone WA14.

SEQ ID NO: 42 is the determined cDNA sequence for clone WA49.

SEQ ID NO: 43 is the determined cDNA sequence for clone WA88.

SEQ ID NO: 44 is the determined cDNA sequence for clone 60s.10.

SEQ ID NO: 45 is the determined cDNA sequence for clone 60s.55.

SEQ ID NO: 46 is the determined cDNA sequence for clone WA11.

SEQ ID NO: 47 is the determined cDNA sequence for clone WA24.

SEQ ID NO: 48 is the determined cDNA sequence for clone WA36.

SEQ ID NO: 49 is the determined cDNA sequence for clone WA5895.

SEQ ID NO: 50 is the determined cDNA sequence for clone WA60.

SEQ ID NO: 51 is the determined cDNA sequence for clone WA79.

SEQ ID NO: 52 is the determined cDNA sequence for clone WA86.

SEQ ID NO: 53 is the determined cDNA sequence for clone WA37.

SEQ ID NO: 54 is the determined cDNA sequence for clone WA70.

SEQ ID NO: 55 is the determined cDNA sequence for clone WA82.

SEQ ID NO: 56 is the determined cDNA sequence for clone WA89.

SEQ ID NO: 57 is the amino acid sequence for clone WA01.

SEQ ID NO: 58 is the amino acid sequence for clone WA14.

SEQ ID NO: 59 is the amino acid sequence for clone WA88.

SEQ ID NO: 60 is the amino acid sequence for clone 60s.10.

SEQ ID NO: 61 is the amino acid sequence for clone 60s.55.

SEQ ID NO: 62 is the amino acid sequence for clone WA11.

SEQ ID NO: 63 is the amino acid sequence for clone WA24

SEQ ID NO: 64 is the amino acid sequence for clone WA36.

SEQ ID NO: 65 is the amino acid sequence for clone WA5895.

SEQ ID NO: 66 is the amino acid sequence for clone WA79.

SEQ ID NO: 67 is the amino acid sequence for clone WA86.

SEQ ID NO: 68 is the amino acid sequence for clone WA37.

SEQ ID NO: 69 is the amino acid sequence for clone WA70.

SEQ ID NO: 70 is the amino acid sequence for clone WA82.

SEQ ID NO: 71 is the amino acid sequence for clone WA89.

SEQ ID NO:72 is the DNA sequence for the coding region of the WA94 antigen, previously described as clone HSP70.94 (SEQ ID NO:11).

SEQ ID NO:73 is the DNA sequence for the coding region of the WA4 antigen, previously described as clone BiP4 (SEQ ID NO:2).

SEQ ID NO:74 is the DNA sequence for the coding region of the WA2 antigen, previously described as clone 60s.2 (SEQ ID NO:1).

SEQ ID NO:75 is the amino acid sequence of the WA4 antigen, previously described as clone BiP4 (SEQ ID NO:2), including an N-terminal Histidine tag.

SEQ ID NO:76 is the amino acid sequence of the WA2 antigen, previously described as clone 60s.2 (SEQ ID NO:1), including an N-terminal Histidine tag.

SEQ ID NO:77 is the amino acid sequence of the WA94 antigen, previously described as clone HSP70.94 (SEQ ID NO:11), including an N-terminal Histidine tag.

SEQ ID NO:78 is the polynucleotide sequence of primer PDM-688, specific for the amplification of WA2.

SEQ ID NO:79 is the polynucleotide sequence of primer PDM-689, specific for the amplification of WA2.

SEQ ID NO:80 is the polynucleotide sequence of primer PDM-690, specific for the amplification of WA4.

SEQ ID NO:81 is the polynucleotide sequence of primer PDM-691, specific for the amplification of WA4.

SEQ ID NO:82 is the polynucleotide sequence of primer PDM-696, specific for the amplification of WA94.

SEQ ID NO:83 is the polynucleotide sequence of primer PDM-697, specific for the amplification of WA94.

SEQ ID NO:84 is the amino acid sequence of an open reading frame with homology to Dynein encoded by clone WA33, referred to as Dynein 33.37 homolog and WA33b.

SEQ ID NO:85 is an extended cDNA sequence of clone WA75.

SEQ ID NO:86 is an amino acid sequence encoded by the extended cDNA sequence of WA75.

SEQ ID NO:87 is the determined cDNA sequence of clone WA29.

SEQ ID NO:88 is the amino acid sequence of clone WA29.

SEQ ID NO:89 is the amino acid sequence of clone WA1 repeat region 1.

SEQ ID NO:90 is the amino acid sequence of clone WA1 repeat region 2.

SEQ ID NO:91 is the consensus amino acid sequence of clone WA1 repeat region 3.

SEQ ID NO:92 is the amino acid sequence of clone WA1 repeat region 4.

SEQ ID NO:93 is the amino acid sequence of clone WA1 repeat region 5.

SEQ ID NO:94 is the amino acid sequence of clone WA1 repeat region 6.

SEQ ID NO:95 is the consensus amino acid sequence of clone WA1 repeat region 7.

SEQ ID NO:96 is the consensus amino acid sequence of clone WA1 repeat region 8.

SEQ ID NO:97 is the amino acid sequence of clone WA1 repeat region 9.

SEQ ID NO:98 is the amino acid sequence of clone WA11 repeat region.

SEQ ID NO:99 is the amino acid sequence of clone WA50a repeat region.

SEQ ID NO:100 is the consensus amino acid sequence of clone WA50b repeat region.

SEQ ID NO:101 is the amino acid sequence of clone WA50b repeat region 1.

SEQ ID NO:102 is the amino acid sequence of clone WA50b repeat region 2.

SEQ ID NO:103 is the amino acid sequence of clone WA50b repeat region 3.

SEQ ID NO:104 is the amino acid sequence of clone WA50b repeat region 4.

SEQ ID NO:105 is the amino acid sequence of clone WA50b repeat region 5.

SEQ ID NO:106 is the amino acid sequence of clone WA50b repeat region 6.

SEQ ID NO:107 is the amino acid sequence of clone WA50b repeat region 7.

SEQ ID NO:108 is the consensus amino acid sequence of clone WA89 repeat region.

### DETAILED DESCRIPTION OF THE INVENTION

As noted above, the present invention is generally directed to compositions and methods for the diagnosis and treatment of Babesia sp. WA1 infection. In one aspect, the compositions of the subject invention include polypeptides that comprise at least one immunogenic portion of a Babesia sp. WA1 antigen, or a variant thereof.

As used herein, the term “polypeptide” encompasses amino acid chains of any length, including full length proteins (i.e., antigens) and fragments thereof, wherein the amino acid residues are linked by covalent peptide bonds. Thus, a polypeptide comprising an immunogenic portion of one of the above antigens may consist entirely of the immunogenic portion, or may contain additional sequences. The additional sequences may be derived from the native Babesia sp. WA1 antigen or may be heterologous, and such sequences may (but need not) be immunogenic.

An “immunogenic portion” of an antigen is a portion that is capable of reacting with sera obtained from an individual infected with Babesia sp. WA1 (i.e., generates an absorbance reading with sera from infected individuals that is at least three standard deviations above the absorbance obtained with sera from uninfected individuals, in a representative ELISA assay described herein). Polypeptides comprising at least an immunogenic portion of one or more Babesia sp. WA1 antigens as described herein may generally be used, alone or in combination, to detect Babesia sp. WA1 in a patient.

Polynucleotides encoding the inventive polypeptides are also provided. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

The compositions and methods of the present invention also encompass variants of the above polypeptides and polynucleotides. Such variants include, but are not limited to, naturally occurring allelic variants of the inventive sequences.

Polynucleotides may comprise a native sequence (i.e., an endogenous sequence that encodes a protein or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native tumor protein. The effect on the immunogenicity of the encoded polypeptide may generally be assessed as described herein. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package. Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, the present invention encompasses polynucleotide and polypeptide sequences, including, e.g., full length sequences, fragments and immunogenic portions, having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. The present invention, in another aspect, provides polypeptide fragments comprising at least about 5, 10, 15, 20, 25, 50, or 100 contiguous amino acids, or more, including all intermediate lengths, of a polypeptide compositions set forth herein. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5×and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

A polypeptide “variant,” as used herein, is a polypeptide that differs from a native protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants encompassed by the present invention include those exhibiting at least about 70%, 75%, 80%, 85%, 90%, 91%, 92%, 93%, 94%, 95%, 96%, 97%, 98%, or 99% or more identity (determined as described above) to the polypeptides disclosed herein.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

In general, Babesia sp. WA1 antigens, and polynucleotides encoding such antigens, may be prepared using any of a variety of procedures. For example, polynucleotides encoding Babesia sp. WA1 antigens may be isolated from a Babesia sp. WA1 genomic or cDNA expression library by screening with sera from individuals infected with Babesia sp. WA1 as described below, and sequenced using techniques well known to those of skill in the art. Polynucleotides encoding Babesia sp. WA1 antigens may also be isolated by screening an appropriate Babesia sp. WA1 expression library with anti-sera (e.g., rabbit) raised specifically against Babesia sp. WA1 antigens.

Antigens may be induced from such clones and evaluated for a desired property, such as the ability to react with sera obtained from an individual infected with Babesia sp. WA1 as described herein. Alternatively, antigens may be produced recombinantly, as described below, by inserting a polynucleotide that encodes the antigen into an expression vector and expressing the antigen in an appropriate host. Antigens may be partially sequenced using, for example, traditional Edman chemistry. See Edman and Berg, Eur. J. Biochem. 80:116-132, 1967.

Polynucleotides encoding antigens may also be obtained by screening an appropriate Babesia sp. WA1 cDNA or genomic DNA library for polynucleotides that hybridize to degenerate oligonucleotides derived from partial amino acid sequences of isolated antigens. Degenerate oligonucleotides for use in such a screen may be designed and synthesized, and the screen may be performed, as described (for example) in Sambrook et al., Molecular Cloning: A Laboratory Manual, Cold Spring Harbor Laboratories, Cold Spring Harbor, N.Y. (and references cited therein). Polymerase chain reaction (PCR) may also be employed, using the above oligonucleotides in methods well known in the art, to isolate a nucleic acid probe from a cDNA or genomic library. The library screen may then be performed using the isolated probe.

Synthetic polypeptides having fewer than about 100 amino acids, and generally fewer than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division, Foster City, Calif., and may be operated according to the manufacturer's instructions.

Immunogenic portions of Babesia sp. WA1 antigens may be prepared and identified using well known techniques, such as those summarized in Paul, Fundamental Immunology, 3d ed., Raven Press, 1993, pp. 243-247 and references cited therein. Such techniques include screening polypeptide portions of the native antigen for immunogenic properties. The representative ELISAs described herein may generally be employed in these screens. An immunogenic portion of a polypeptide is a portion that, within such representative assays, generates a signal in such assays that is substantially similar to that generated by the full length antigen. In other words, an immunogenic portion of a Babesia sp. WA1 antigen generates at least about 20%, and preferably about 100%, of the signal induced by the full length antigen in a model ELISA as described herein.

Portions and other variants of Babesia sp. WA1 antigens may be generated by synthetic or recombinant means. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Recombinant polypeptides containing portions and/or variants of a native antigen may be readily prepared from a polynucleotide encoding the polypeptide using a variety of techniques well known to those of ordinary skill in the art. For example, supernatants from suitable host/vector systems which secrete recombinant protein into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant protein.

Any of a variety of expression vectors known to those of ordinary skill in the art may be employed to express recombinant polypeptides as described herein. Expression may be achieved in any appropriate host cell that has been transformed or transfected with an expression vector containing a polynucleotide that encodes a recombinant polypeptide. Suitable host cells include prokaryotes, yeast and higher eukaryotic cells. Preferably, the host cells employed are E. coli, yeast or a mammalian cell line, such as COS or CHO. The polynucleotides expressed in this manner may encode naturally occurring antigens, portions of naturally occurring antigens, or other variants thereof.

In general, regardless of the method of preparation, the polypeptides and polynucleotides disclosed herein are prepared in an isolated, substantially pure, form. Preferably, the polypeptides and polynucleotides are at least about 80% pure, more preferably at least about 90% pure and most preferably at least about 99% pure.

In a further aspect, the present invention provides fusion proteins comprising either a first and a second inventive polypeptide, or an inventive polypeptide and a known polypeptide, together with variants of such fusion proteins. The fusion proteins of the present invention may also include a linker peptide between the polypeptides.

A polynucleotide encoding a fusion protein of the present invention is constructed using known recombinant DNA techniques to assemble separate polynucleotides encoding, for example, the first and second polypeptides into an appropriate expression vector. The 3′ end of a polynucleotide encoding the first polypeptide is ligated, with or without a peptide linker, to the 5′ end of a polynucleotide encoding the second polypeptide so that the reading frames of the sequences are in phase to permit mRNA translation of the two polynucleotides into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8562, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric hindrance.

In another aspect, the present invention provides methods for using polypeptides comprising an immunogenic portion of a Babesia sp. WA1 antigen to diagnose babesiosis. In this aspect, methods are provided for detecting Babesia sp. WA1 infection in a biological sample, using one or more of the above polypeptides.

As used herein, a “biological sample” is any antibody-containing sample obtained from a patient. Preferably, the sample is whole blood, sputum, serum, plasma, saliva, cerebrospinal fluid or urine. More preferably, the sample is a blood, serum or plasma sample obtained from a patient. The polypeptides are used in an assay, as described below, to determine the presence or absence of antibodies to the polypeptide(s) in the sample, relative to a predetermined cut-off value. The presence of such antibodies indicates previous sensitization to Babesia sp. WA1 antigens which may be indicative of babesiosis.

In embodiments in which more than one polypeptide is employed, the polypeptides used are preferably complementary (i.e., one component polypeptide will tend to detect infection in samples where the infection would not be detected by another component polypeptide). Complementary polypeptides may generally be identified by using each polypeptide individually to evaluate serum samples obtained from a series of patients known to be infected with Babesia sp. WA1. After determining which samples test positive (as described below) with each polypeptide, combinations of two or more polypeptides may be formulated that are capable of detecting infection in most, or all, of the samples tested.

A variety of assay formats are known to those of ordinary skill in the art for using one or more polypeptides to detect antibodies in a sample. See, e.g., Harlow and Lane, Antibodies: A Laboratory Manual, Cold Spring Harbor Laboratory, 1988, which is incorporated herein by reference. In a preferred embodiment, the assay involves the use of polypeptide immobilized on a solid support to bind to and remove the antibody from the sample. The bound antibody may then be detected using a detection reagent that contains a reporter group. Suitable detection reagents include antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the antigen may be attached. For example, the solid support may be a test well in a microtiter plate, or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example in U.S. Pat. No. 5,359,681.

The polypeptides may be bound to the solid support using a variety of techniques known to those of ordinary skill in the art. In the context of the present invention, the term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide ranging from about 10 ng to about 1 μg, and preferably about 100 ng, is sufficient to bind an adequate amount of antigen.

Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to supports having an appropriate polymer coating using benzoquinone or by condensation of an aldehyde group on the support with an amine and an active hydrogen on the polypeptide (see, e.g., Pierce Immunotechnology Catalog and Handbook, 1991, at A12-A13).

In certain embodiments, the assay is an enzyme linked immunosorbent assay (ELISA). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin (BSA) or Tween 20™ (Sigma Chemical Co., St. Louis, Mo.) may be employed. The immobilized polypeptide is then incubated with the sample, and antibody is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody within a Babesia sp. WA1-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., and Pierce, Rockford, Ill.).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-Babesia sp. WA1 antibodies in the sample, the signal detected from the reporter group that remains bound to the solid support is generally compared to a signal that corresponds to a predetermined cut-off value. In one preferred embodiment, the cut-off value is the average mean signal obtained when the immobilized antigen is incubated with samples from an uninfected patient. In general, a sample generating a signal that is three standard deviations above the predetermined cut-off value is considered positive for babesiosis. In an alternate preferred embodiment, the cut-off value is determined using a Receiver Operator Curve, according to the method of Sackett et al., Clinical Epidemiology: A Basic Science for Clinical Medicine, Little Brown and Co., 1985, pp. 106-107. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for babesiosis.

In a related embodiment, the assay is performed in a rapid flow-through or strip test format, wherein the antigen is immobilized on a membrane, such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of anti-Babesia sp. WA1 antibodies in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when the biological sample contains a level of antibodies that would be sufficient to generate a positive signal in an ELISA, as discussed above. Preferably, the amount of polypeptide immobilized on the membrane ranges from about 25 ng to about 1 μg, and more preferably from about 50 ng to about 500 ng. Such tests can typically be performed with a very small amount (e.g., one drop) of patient serum or blood.

Of course, numerous other assay protocols exist that are suitable for use with the polypeptides of the present invention. The above descriptions are intended to be exemplary only.

In yet another aspect, the present invention provides antibodies to the polypeptides of the present invention. Antibodies may be prepared by any of a variety of techniques known to those of ordinary skill in the art. See, e.g., Harlow and Lane, Antibodies: A Laboratory Manual, Cold Spring Harbor Laboratory, Cold Spring Harbor, N.Y., 1988. In one such technique, an immunogen comprising the antigenic polypeptide or epitope is initially injected into any of a wide variety of mammals (e.g., mice, rats, rabbits, sheep and goats). The polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Antibodies may be used in diagnostic tests to detect the presence of Babesia sp. WA1 antigens using assays similar to those detailed above and other techniques well known to those of skill in the art, thereby providing a method for detecting Babesia sp. WA1 infection in a patient.

The presence of Babesia sp. WA1 infection may also, or alternatively, be detected based on the level of mRNA encoding a Babesia sp. WA1-specific protein in a biological sample. For example, at least two oligonucleotide primers may be employed in a polymerase chain reaction (PCR) based assay to amplify a portion of a Babesia sp. WA1-specific cDNA derived from a biological sample, wherein at least one of the oligonucleotide primers is specific for (i.e., hybridizes to) a polynucleotide encoding the Babesia sp. WA1 protein. The amplified cDNA is then separated and detected using techniques well known in the art, such as gel electrophoresis. Similarly, oligonucleotide probes that specifically hybridize to a polynucleotide encoding a Babesia sp. WA1 protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a Babesia sp. WA1 protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule that is complementary to polynucleotide disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

Primers or probes may thus be used to detect Babesia sp. WA1-specific sequences in biological samples, preferably sputum, blood, serum, saliva, cerebrospinal fluid or urine. Oligonucleotide primers and probes may be used alone or in combination with each other.

In another aspect, the present invention provides methods for using one or more of the above polypeptides or fusion proteins (or polynucleotides encoding such polypeptides) to induce protective immunity against Babesia sp. WA1 infection in a patient. As used herein, a “patient” refers to any warm-blooded animal, preferably a human. A patient may be afflicted with a disease, or may be free of detectable disease and/or infection. In other words, protective immunity may be induced to prevent or treat babesiosis.

In this aspect, the polypeptide, fusion protein or polynucleotide is generally present within a pharmaceutical composition, or a vaccine or immunogenic composition. Pharmaceutical compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant or a liposome (into which the polypeptide is incorporated). Such pharmaceutical compositions and immunogenic compositions may also contain other Babesia sp. WA1 antigens, either incorporated into a combination polypeptide or present as a separate polypeptide.

Alternatively, an immunogenic composition may contain a polynucleotide encoding one or more polypeptides or fusion proteins as described above, such that the polypeptide is generated in situ. In such immunogenic compositions, the polynucleotide may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the polynucleotide may be introduced using a viral expression system (e.g., vaccinia or other pox virus, retrovirus, or adenovirus), which may involve the use of a non-pathogenic (defective), replication competent virus. Techniques for incorporating polynucleotides into such expression systems are well known to those of ordinary skill in the art. The polynucleotide may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In a related aspect, a DNA vaccine as described above may be administered simultaneously with or sequentially to either a polypeptide of the present invention or a known Babesia antigen. For example, administration of a polynucleotide encoding a polypeptide of the present invention, either “naked” or in a delivery system as described above, may be followed by administration of an antigen in order to enhance the protective immune effect of the vaccine, or immunogenic composition.

Routes and frequency of administration, as well as dosage, will vary from individual to individual. In general, the pharmaceutical compositions and immunogenic compositions may be administered by injection (e.g., intracutaneous, intramuscular, intravenous or subcutaneous), intranasally (e.g., by aspiration) or orally. Between 1 and 3 doses may be administered for a 1-36 week period. Preferably, 3 doses are administered, at intervals of 3-4 months, and booster vaccinations may be given periodically thereafter. Alternate protocols may be appropriate for individual patients. A suitable dose is an amount of polypeptide or polynucleotide that, when administered as described above, is capable of raising an immune response in an immunized patient sufficient to protect the patient from infection with Babesia sp. WA1 for at least 1-2 years. In general, the amount of polypeptide present in a dose (or produced in situ by the polynucleotide in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 mL to about 5 mL.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of this invention to enhance the immune response. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aliminium hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants. In certain embodiments, the inventive immunogenic compositions include an adjuvant capable of eliciting a predominantly Th-1 type response. Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corp. (Hamilton, Mont.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555 and WP 99/33488. Immunostimulatory DNA sequences are also described, for example, by Sato et al., Science 273:352, 1996. Another preferred adjuvant is a saponin, preferably QS21 (Aquila, United States), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham. Rixensart. Belgium). Detox (Corixa. Hamilton, Mont.). RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

The following Examples are offered by way of illustration and not by way of limitation.

### EXAMPLES

### Example 1

### Isolation of Polynucleotides Encoding Babesia sp. WA1 Antigens

This example illustrates the isolation of polynucleotides encoding Babesia sp. WA1 antigens by screening an unamplified Babesia sp. WA1 cDNA expression library with a human patient sera pool.

The cDNA expression library was constructed as follows. Infection with Babesia sp. WA1 was established by intraperitoneal inoculation of 500 ul of cryopreserved, infected hamster blood into 3-week-old 50 g female Golden Syrian hamsters (SASCO; Charles River, Wilmington, Mass.). Infection was monitored by use of Giemsa-stained or acridine orange-stained blood smears over a 2-week period. Blood was harvested into EDTA tubes and the red cells were separated via centrifugation. The nucleic acids were isolated from the red cell fraction and separated on a CsCl gradient. The RNA was re-extracted with phenol/chloroform and precipitated with ethanol. The poly A mRNA was isolated using a kit from Promega (Madison, Wis.) for small scale mRNA isolation and cDNA was made from the polyA mRNA by oligo dT priming. The cDNA fragments were blunt ended and then ligated to EcoRI adapters (Stratagene, La Jolla, Calif.). The inserts were size selected with a Sephacryl S-400-HR column (Sigma Chemical Co, St. Louis, Mo.) and then ligated into the Lambda ZAP II vector (Stratagene). The ligation mix was packaged with Gigapack II Gold packaging extract (Stratagene).

Before screening, the serum was adsorbed with E. coli proteins on nitrocellulose filters. The library was plated on eleven large Petri plates (150 mm×15 mm) at a concentration of approximately 20,000 plaques/plate (total number of plaques screened=2.2×10⁵). The plaques were transferred to nitrocellulose filters and then processed using established protocols with adsorbed SCID-mouse s e r a as the primary antibody and goat anti-mouse (IgG, IgA, IgM HPL), alkaline phosphatase conjugated, secondary antibody to visualize positive plaques. Ninety-five plaques were picked upon the first screening of the library. These plaques were then processed and replated for secondary screens and in some cases tertiary screens, again using the SCID sera as the primary antibody. Eighty-three clones were confirmed as positive with the secondary and tertiary screens. These plaques were processed according to the protocols developed by Stratagene for their ZAP II vector for excision of the insert and subsequent cloning into their SOLR strain of E. coli (Stratagene). Individual clones were grown overnight in appropriate media. A small portion of the culture was frozen down in glycerol to serve as future stock and the remainder of the culture was processed to extract the plasmid DNA for analysis.

The DNA from the inserts in each clone was sequenced in both directions. Many of the clones were identical or homologous to other isolated clones and were grouped. These groups contain what are believed to be separate mRNAs from multi-copy genes within the genome and/or truncated cDNAs from a single gene. The determined cDNA sequence for the isolated clones are provided in SEQ ID NO: 1-23, and 40-56 with the corresponding amino acid sequences and database analyses being shown in Table 1 below. The clones of SEQ ID NO: 12, 15 and 22 represent partial cDNA sequences. The remaining cDNA sequences are believed to either be full-length and/or to contain an open reading frame.

TABLE 1

| | | Amino | |
|---|---|---|---|
| | cDNA | acid | |
| | SEQ ID | SEQ | Homology to known |
| Clone | NO: | ID NO: | sequences |
| WA1 | | | |
| family | | | |
| WA1 | 14 | 30 | No significant homologies |
| WAO1 | 40 | 57 | |
| WA14 | 41 | 58 | |
| WA49 | 42 | — | |
| WA88 | 43 | 59 | |
| BiP | | | HSP70-related family, |
| family: | | | referred to as BiP |
| BiP4 | 2 | 38 | (immunoglobin binding |
| BiP57 | 3 | 37 | protein) |
| BiP63 | 4 | — | |
| BiP80 | 5 | — | |
| WA5 | 19 | 26 | No significant homologies |
| WA6 | | | |
| family: | | | |
| WA6 | 20 | — | No significant homologies |
| WA16 | 13 | 31 | |
| WA38 | 17 | 28 | |
| HSP70 | | | |
| family: | | | |
| HSP70.19 | 6 | 36 | Homology to the heat shock |
| HSP70.22 | 7 | — | protein HSP70 |
| HSP70.28 | 8 | 35 | |
| HSP70.61 | 9 | — | |
| HSP70.68 | 10 | 34 | |
| HSP70.94 | 11 | 33 | |
| 60s.2 | | | |
| family | | | |
| 60s.2 | 1 | 39 | Homology to family of |
| 60s.10 | 44 | 60 | proteins called 60s Acidic |
| 60s.55 | 45 | 61 | Ribosomal proteins |
| WA11 | | | |
| family: | | | |
| WA11 | 12 (partial) | 32, 62 | No significant homologies |
| | 46 (full- | | |
| | length) | | |
| WA24 | 15 (Partial) | 63 | |
| | 47 (full- | | |
| | length) | | |
| WA36 | 48 | 64 | |
| WA5895 | 49 | 65 | |
| WA60 | 50 | — | |
| WA79 | 51 | 66 | |
| WA86 | 52 | 67 | |
| WA33 | | | |
| family: | | | |
| WA33 | 16 | 29 | No significant homologies |
| WA37 | 53 | 68 | |
| WA41 | | | |
| family: | | | |
| WA41 | 18 | 27 | No significant homologies |
| WA76 | 23 | — | |
| WA50 | | | |
| family: | | | |
| WA74 | 21 | 25 | No significant homologies |
| WA75 | | | |
| family: | | | |
| WA75 | 22 | 24 | No significant homologies |
| WA70 | | | |
| family: | | | |
| WA70 | 54 | 69 | No significant homologies |
| WA82 | | | |
| family: | | | |
| WA82 | 55 | 70 | No significant homologies |
| WA89 | | | |
| family: | | | |
| WA89 | 56 | 71 | No significant homologies |

### Example 2

### Expression of Recombinant WA-1 Antigens

The open reading frame of WA2, previously described as 60s.2 (the sequence of which is disclosed in SEQ ID NO:39) was amplified by PCR using the PCR primers PDM-688 (SEQ ID NO:78) and PDM-589 (SEQ ID NO:79). The PCR mixture contained the following components: 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene) and 50 ng DNA. Amplification was carried out under the following reaction conditions: denaturation at 96° C. was performed for 2 min, followed by 40 cycles of 96° C. for 20 sec. 58° C. for 15 sec and 72° C. for 4 min, with a final extension cycle of 72° C. for 4 min. The PCR product was digested with EcoRI and cloned into a modified pET28 vector with a His tag in-frame on the 5′ end, which had been digested with Eco721 and EcoRI. The correct construct was confirmed through sequence analysis and transformed into BLR(DE3)pLysS E. coli cells and BLR(DE3) E. coli cells which were cotransformed with the CodonPlus RIL. The amino acid sequence of the recombinant WA2 protein, including His tag, is provided in SEQ ID NO: 76, with the cDNA sequence of the coding region being provided in SEQ ID NO: 74.

The open reading frame of WA4, previously described as BiP4 (the sequence of which is disclosed in SEQ ID NO:38) was amplified by PCR using the PCR primers PDM-690 (SEQ ID NO:80) and PDM-691 (SEQ ID NO:81). The PCR mixture contained the following components: 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene) and 50 ng DNA. Amplification was carried out under the following reaction conditions: denaturation at 96° C. was performed for 2 min, followed by 40 cycles of 96° C. for 20 sec, 57° C. for 15 sec and 72° C. for 4 min, with a final extension cycle of 72° C. for 4 min. The PCR product was digested with EcoRI and cloned into a modified pET28 vector with a His tag in-frame on the 5′ end, which had been digested with Eco721 and EcoRI. The correct construct was confirmed through sequence analysis and transformed into BLR(DE3)pLysS E. coli cells and BLR(DE3) E. coli cells which were cotransformed with the CodonPlus RIL. The amino acid sequence of the recombinant WA4 protein, including His tag, is provided in SEQ ID NO: 75, with the cDNA sequence of the coding region being provided in SEQ ID NO: 73.

The open reading frame of WA-94, previously described as HSP70.94 (the sequence of which was disclosed in SEQ ID NO:33) was amplified by PCR using the PCR primers PDM-696 (SEQ ID NO:82) and PDM-697 (SEQ ID NO:83). The PCR mixture contained the following components: 10 μl of 10×Pfu buffer (Stratagene), 1 μl of 10 mM dNTPs, 2 μl each of the PCR primers at 10 μM concentration, 83 μl water, 1.5 μl Pfu DNA polymerase (Stratagene) and 50 ng DNA. Amplification was carried out under the following reaction conditions: denaturation at 96° C. was performed for 2 min, followed by 40 cycles of 96° C. for 20 sec, 62° C. for 15 sec and 72° C. for 4 min, with a final extension cycle of 72° C. for 4 min. The PCR product was digested with XhoI and cloned into a modified pET28 vector with a His tag in-frame on the 5′ end, which had been digested with Eco721 and XhoI. The correct construct was confirmed through sequence analysis and transformed into BLR(DE3)pLysS E. coli cells and BLR(DE3) E. coli cells which were cotransformed with the CodonPlus RIL. The amino acid sequence of the recombinant WA94 protein, including His tag, is provided in SEQ ID NO: 77, with the cDNA sequence of the coding region being provided in SEQ ID NO: 72.

### Example 3

### Characterization of Babesia sp. WA1 Antigens

Additional polynucleotide and polypeptide sequence analysis was performed on the Babesia Sp. WA1 clones identified as described in Example 1. This analysis revealed the presence of additional open reading frames and sequence homologies associated with these antigens. In addition, polypeptide repeats and predicted functional domains were identified in the amino acid sequences of a number of the Babesia Sp. WA1 clones.

The cDNA sequence corresponding to clone WA29, which was identified in the screen described in Example 1, is provided in SEQ ID NO:87, and the corresponding polypeptide sequence encoded by SEQ ID NO:87 is provided in SEQ ID NO:88. The amino acid sequence of SEQ ID NO:88 shows homology to various XPG/FEN/RAD2 endonucleases.

Further analysis of the polynucleotide sequence of clone WA33 (SEQ ID NO:16) revealed the presence of an additional encoded open reading frame. The amino acid sequence of this open reading frame is provided in SEQ ID NO:84 and exhibits homology to various dynein homologs.

Additional nucleic acid sequencing was performed on the WA75 cDNA clone, and the resulting polynucleotide sequence is set forth in SEQ ID NO:85. The predicted amino acid sequence encoded by the WA75 polynucleotide sequence set forth in SEQ ID NO:85 is provided in SEQ ID NO:86.

All WA1 clone nucleotide and polypeptide sequences were analyzed and searches were performed against the GenBank, EST mouse, and GENESEQ databases using the BLST programs (blastn, blastp, blastx, or tblastx). Predicted polypeptides were analyzed using the PSORT and PSORT II programs (Human Genome Center, IMS, Tokyo, Japan), Identify (Stanford University, Palo Alto, Calif.), Signal P V1.1 program (Nielsen, H. et al., Protein Engineering 12:3-9 (1999)), Tmpred—Prediction of transmembrane regions and orientation, and the Pfam program.

Several of the WA1 clones were found to contain predicted signal sequences. The polypeptide sequences of the WA1, WA5, WA11, and WA41 clones all contain predicted cleavable signal sequences, while the polypeptide sequences of the BiP and WA75 clones contain predicted uncleavable signal sequences. In addition to their development for a traditional ELISA, wherein recombinant proteins are used to detect reactive antibodies in serum, the presence of signal sequences within these clones makes them particularly useful in the development of a diagnostic sandwich ELISA, relying on the detection of antigen in serum using monoclonal antibodies.

Predicted membrane domains were identified in a number of clones and clone families. The WA1 family of clones is predicted to be a type IIIa membrane protein, with a potential helix-turn-helix motif. WA5, WA33b, and WA82 are predicted to be type 11 membrane proteins, with WA33b also containing a region of homology to dynein light chain. In addition, clone WA41 is predicted to be a type la membrane prtoein.

Amino acid repeat sequences were identified in a number of the WA1 clones. For example, an approximately 80 amino acid residue repeat was identified in WA1 family members, a 12 amino acid repeat sequence was identified in WA11, a 23 amino acid repeat was identified in WA75, a 7-8 amino acid repeat was identified in WA74, and a 6 amino acid repeat was identified in WA89. The repeat sequences identified in WA1 family members are provided in SEQ ID NOs:89-97, and the repeat sequence of WA11 is provided in SEQ ID NO:98. In addition, the repeat sequence identified in the first identified open reading frame of WA50 (WA50a) is provided in SEQ ID NO:99. The consensus repeat sequence identified in the second identified open reading frame of WA50 (WA50b) is provided in SEQ ID NO:100, with specific repeat sequences of WA50b set forth in SEQ ID NOs:101-107. Also, the consensus repeat sequence identified in WA89 is provided in SEQ ID NO:108. These repeat sequences are predicted to be antigenic epitopes that may be used in the development of diagnostic or therapeutic reagents and methods according to the invention.

A number of WA1 clones showed homology to known and, in some cases, well-characterized protein families. A summary of these homologies is provided below in Table 2. Of particular note, SEQ ID NO:84 shows homology to dynein light chain polypeptides. Since dynein polypeptides are known to have immunomodulatory effects, this clone should also be useful in the development of an adjuvant.

TABLE 2
Summary of homologies of Babesia sp. WA1 clone peptide
sequences with known sequences

| Name of | Percent | | |
|---|---|---|---|
| family | Identity | Sequence ID: | Description: |
| WA1 family: | 30 (over | 310893 | Membrane protein from Theileria |
| (SEQ ID NO:30) | 386bp) | | parva |
| | 27 (over | 310892 | Glutamine rich membrane protein |
| | 143bp) | | from T. parva |
| | 39 (over | 310892 | Glutamine rich membrane protein |
| | 176bp) | | from T. parva |
| HSP70/BiP | 64 (over | 6682358 | Heat shock protein 70 precursor |
| homolog: | 613 aa | | from Toxoplasma gondii |
| (SEQ ID NO:38) | 64 (over | 1037175 | Immunoglobin heavy chain |
| | 605 aa) | | binding protein (BiP) from Eimeria |
| | | | tenella |
| WA5: | Nothing significant over 20% identity | | |
| (SEQ ID NO:26) | | | |
| WA6 (seq: | 24 (over | 15221899 | Hypothetical protein from |
| WA16 final): | 458bp) | | Arabidopsis thaliana |
| (SEQ ID NO:31) | 24 (over | AAW24790 | Liver stage antigen-3 from |
| | 518 bp) | | Plasmodium falciparum |
| HSP70 | 86 (over | 6492133 | Heat shock protein 70 from |
| homolog: | 611 aa | | Babesia bovis |
| (SEQ ID NO:33) | 85 (over | 1100899 | Heat shock protein 70 from |
| | 611 aa) | | Theileria parva |
| 60s acidic | | | |
| ribosomal | 54 (over | 14579677 | Ribosomal phosphoprotein (P0) |
| protein | 306 aa | | from Toxoplasma gondii |
| (aka P0) | 51 (over | 13774516 | Ribosomal protein P0 from |
| homolog: | 303 aa | | Eimeria tenella |
| (SEQ ID NO:39) | 49 (over | 4191736 | Acidic ribosomal phosphoprotein |
| | 311 aa | | P0 from P. falciparum |
| WA11 | 32 (over | 501027 | ORF2 from Trypanosoma brucei |
| family: | 341bp)¹ | | |
| (SEQ ID NO:62) | 35 (over | 13357558 | Ureaplasma urealyticum, |
| | 398bp) | | complete genome |
| | 37 (over | 160218 | Circumsporozoite protein (CSP) |
| | 356bp) | | from Plasmodium knowlesi |
| RAD-2 | 35 (over | 16804952 | RAD2 endonuclease from |
| homolog: | 878bp) | | Plasmodium faliciparum |
| (aka XPG1, | 33/38(65 | AAW92507 | Yeast delta-RAD2 protein |
| FEN-1) | 9bp/ | | |
| (SEQ ID NO:88) | 233bp) | | |
| | 26/33 | AAW92505 | Mouse FEN-1 protein |
| | (833 bp/ | | |
| | 233bp) | | |
| WA33a: | 22 (over | 4503509 | Eukaryotic translation initiation |
| (SEQ ID NO:29) | 434 aa) | | factor 3 from Homo sapiens |
| | 21 (over | 6686292 | Eurkaryotic translation initiation |
| | 434 aa | | factor 3 from Mus musculus |
| WA33b | 64 (over | 2811014 | Dynein light chain LC6 from |
| (Dynein): | 87 aa | | Anthocidaris crassispina |
| (SEQ ID NO:84) | 67 (over | 2494222 | Probably dynein light chain 1 from |
| | 84 aa | | Caenorhabditis elegans |
| | 64 (over | AAW56785 | Protein inhibitor of neuronal nitric |
| | 84 aa | | oxide synthase-1 (PIN-1) |
| WA41: | No significant homologies. | | |
| (SEQ ID NO:27) | | | |
| WA50a: | 27 (over | 1710805 | RTOA protein (rtoA gene product) |
| (SEQ ID NO:24) | 656bp) | | from Dictyostelium Discoideum |
| WA50b: | 23 (over | 16604617 | Putative myosin heavy chain from |
| (SEQ ID NO:25) | 368bp) | | Arabidopsis thaliana |
| WA64: | 26 (over | 15810454 | Eukaryotic initiation factor 4 from |
| (SEQ ID NO:69) | 324 aa | | Arabidopsis thaliana |
| | 28 (over | 3056722 | Eurkaryotic initiation factor 4, |
| | 300 aa) | | eIF4, from Drosophila |
| | | | melanogastor |
| WA82: | 27 (over | 15823627 | P18 protein from Babesia gibsoni |
| (SEQ ID NO:70) | 896bp) | | |
| | 27 (over | 6017001 | Thrombospondin-related |
| | 806bp) | | adhesive protein from Neospora |
| | | | caninum |
| WA89: | No significant homologies. | | |
| (SEQ ID NO:71) | | | |

### Example 4

### Reactivity of Various Sera to Recombinant Babesia sp. WA1 Proteins

Sera from patients infected with Babesi sp. WA1, Babesia sp. CA, Babesia microti, Toxoplasma, as well as sera from patients with high WA1 IFA titer but unconfirmed infection and random donors were screened by ELISA analysis using the bacterially expressed recombinant His-tagged WA1 polypeptides described in Example 2. ELISA analysis was performed as described in Lodes, M. J. et al., Infect. Immun. 68:2783-90 (2000) using 200 ng of recombinant protein to coat each well and patient sera at a dilution of 1:100.

The results of the ELISA analysis are presented below in Table 4. Of note, Babesia sp. CA-positive sera did not show the same reactivity as WA1-positive sera. In addition, most of the Babesia microti-positive sera showed reactivity to WA1 HSP70 but not the closely related WA1 GRP78.

These experiments confirm the immunogenicity of various WA1 polypeptides and demonstrate that such polypeptides, including recombinantly expressed polypeptides, are useful in the detection of infection by specific species of Babesia and some are capable of distinguishing between infection by different Babesia species. In addition, the experiments described above support the use of the identified WA1 antigens as targets for vaccine and other immunotherapeutic approaches.

TABLE 3
Reactivity of various sera to recombinant Babesia sp. WA1 proteins

| | | | P0 |
|---|---|---|---|
| | | | (acidic ribosomal) |
| | HSP 70 | GRP78 | protein |
| | (SEQ ID NO:77) | (SEQ ID NO:75) | (SEQ ID NO:76) |
| Confirmed WA1 | 3/3 | 2/3* | 0/3* |
| patient sera | | | |
| Confirmed Babesia | 2/5 | 0/5 | 1/5 |
| sp CA patient sera | | | |
| Confirmed Babesia | 23/25* | 5/25 | 19/25 |
| microti positive | | | |
| patient sera | | | |
| Confirmed | 0/10 | 0/10 | (5/10) |
| Toxoplasma positive | | | |
| patient sera | | | |
| Patient sera with | 1/37 | 4/37 | 8/37 |
| high WA1 IFA titer- | | | |
| unconfirmed | | | |
| infection | | | |
| Random donors | 0/31 | 1/31 | 0/31 |

*Remaining “negative”sera showed reactivity levels at cut-off point, therefore, they could be considered borderline positive.

Although the present invention has been described in some detail by way of illustration and example for purposes of clarity of understanding, changes and modifications can be carried out without departing from the scope of the invention which is intended to be limited only by the scope of the appended claims.

1. An isolated polynucleotide comprising a sequence selected from the group consisting of:
(a) sequences provided in SEQ ID NO: 1-23, 40-56, 72-74, 85 and 87;
(b) complements of the sequences provided in SEQ ID NO: 1-23, 40-56, 72-74, 85 and 87;
(c) sequences that hybridize to a sequence provided in SEQ ID NO: 1-23, 40-56, 72-74, 85 and 87, under moderately stringent conditions;
(d) sequences having at least 75% identity to a sequence of SEQ ID NO: 1-23, 40-56, 72-74, 85 and 87;
(e) sequences having at least 90% identity to a sequence of SEQ ID NO: 1-23, 40-56,72-74, 85 and 87; and
(f) degenerate variants of a sequence provided in SEQ ID NO: 1-23, 40-56, 72-74, 85 and 87.

2. An isolated polypeptide comprising an amino acid sequence selected from the group consisting of:
(a) SEQ ID NO: 24-39, 57-71,75-77, 84, 86 and 88;
(b) sequences encoded by a polynucleotide of claim 1;
(c) sequences having at least 70% identity to a sequence encoded by a polynucleotide of claim 1; and
(d) sequences having at least 90% identity to a sequence encoded by a polynucleotide of claim 1.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A fusion protein comprising at least an immunogenic portion of a polypeptide according to claim 2.

7. An oligonucleotide that hybridizes to a sequence recited in SEQ ID NO: 1-23, 40-56, 72-74, 85 and 87 under moderately stringent conditions.

8. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5; and
(d) fusion proteins according to claim 6.

9. A method for stimulating an immune response in a patient, comprising administering to the patient a composition of claim 8.

10. A method for the treatment of Babesia infection in a patient, comprising administering to the patient a composition of claim 8.

11. A method for determining the presence of Babesia infection in a patient, comprising the steps of:
(a) obtaining a biological sample from the patient;
(b) contacting the biological sample with an oligonucleotide according to claim 7;
(c) detecting in the sample an amount of a polynucleotide that hybridizes to the oligonucleotide; and
(d) comparing the amount of polynucleotide that hybridizes to the oligonucleotide to a predetermined cut-off value, and therefrom determining Babesia infection in the patient.

12. A diagnostic kit comprising at least one oligonucleotide according to claim 7.

13. A diagnostic kit comprising at least one antibody according to claim 5 and a detection reagent, wherein the detection reagent comprises a reporter group.

14. A method for detecting Babesia infection in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining Babesia infection in the patient.

15. A method for detecting Babesia infection in a patient, comprising:
(a) obtaining a biological sample from the patient;
(b) contacting the sample with at least one polypeptide according to claim 2; and
(c) detecting the presence of antibodies that bind to the polypeptide.

16. A method for detecting Babesia infection in a patient, comprising:
(a) obtaining a biological sample from the patient;
(b) contacting the sample with a fusion protein according to claim 6; and
(c) detecting the presence of antibodies that bind to the fusion protein.

17. A diagnostic kit comprising
(a) at least one polypeptide according to claim 2; and
(b) a detection reagent.

18. A diagnostic kit comprising:
(a) at least one fusion protein according to claim 6; and
(b) a detection reagent.
